(19) United States
(12) Patent Application Publication
(10) Pub. No.: US 2010/0197026 A1
(43) Pub. Date: Aug. 5, 2010

(54) SOLUTION FOR DETERMINING OVULATORY PERIOD IN WOMEN FROM A TEST TUBE OF URINE

(76) Inventors: Imrich Beller, Topol'cany (SK); Robert Beller, Nitra (SK)

Correspondence Address: Pearl Cohen Zedek Latzer, LLP, 1500 Broadway, 12th Floor, New York, NY, 10036, US

(21) Appl. No.: 12/669,354

(22) PCT Filed: Jul. 14, 2008
(86) PCT No.: PCT/SK2008/000009

§ 371 Date: Jan. 15, 2010

(30) Foreign Application Priority Data
Jul. 17, 2007 (SK) PP 98-2007

(51) Int. Cl. G01N 33/48; G01N 33/00

(52) U.S. Cl. 436/65; 252/408.1

(57) ABSTRACT

The solution covered by the invention contains 0.5 to 9.0% by mass of hydrate salt of chloride anion Cl⁺ with a chemical element of bivalent kation of cobalt or copper, magnesium, manganese, zinc, calcium or a monovalent kation of sodium, potassium; 0.5 to 10.5% by volume of oxalic acid, formic acid, acetic acid, butyric acid, citric acid, lactic acid, hydrofluoric acid, boric acid, tartaric acid; 0.2 to 8.0% by mass of casein and 80.5 to 98.5% by volume of water. The overall pH of the solution is 0.8 to 3.5%.

### TECHNICAL FIELD

The invention refers to a solution, which causes a direct bond of free SO₄²⁻ anions in a sample of urine to the anion, corresponding cation, acid and carrier (proteins), added by the solution covered by the invention, in an acidic environment.

### BACKGROUND ART

The solution covered by the invention can be characterised in brief as an indirect contraceptive. In comparison to any other existing contraceptive methods and forms, of which approximately 95% achieves their contraceptive effect by intervening with the female organism, resulting in certain direct effects on female health or even on their children (mainly in synthetic hormonal preparations, intrauterine body), the solution covered by the invention shows properties of an ideal contraceptive in terms of effectiveness.

The term “contraception” generally refers to any method, form, application and usage aimed at preventing discharged sperm from fertilising a released egg during a sexual intercourse at the time of ovulation.

Thinking about drives affecting sexuality, it is necessary to realise that sexuality is the fundamental biological capacity to preserve species existence, which is firmly encoded as a piece of genetic information in every living organism. In humans, this capacity is complemented by a very rare ability—the ability of intensive and productive thinking with every kind of emotional characteristics and their manifestations. This is the basis and a presumption that sexuality is a general biological strive of every human with healthy reproduction organs and abilities.

### Contraceptive Methods

At present, there are only three forms of intentional birth control: sexual abstinence, induced abortion and contraception.

Assessment of contraception and its existing forms shows that different contraceptive methods have been used for thousands years depending always on the level of expert (scientific) knowledge available in the given period of time. One has to admit, however, that neither our present scientific and technical level provided us with any optimum method of contraception.

The optimum contraceptive would be a choice that would reliably protect the woman against pregnancy without any health risks or side effects (in children, too) and would not interfere with the sexual intercourse. Effects of such contraception should be completely reversible, without any medical check-ups and with easy administration.

Indeed, there are objections to some forms and methods of contraception in various aspects, but the opinion of the World Health Organisation is clear: only reliable and safe contraception can avert the horrific vision of an overcrowded planet that might have catastrophic consequences for the mankind with all the accompanying effects in the process of its gradual intensification.

Based on a calculation done by the UN, preventing the tragedy of overcrowding our planet EARTH requires a dramatic decrease of fertility in developing countries in particular.

At present, we know and have available a wider range of contraceptive sets and contraceptive methods.

### Natural Contraception

These methods derive from and are applied without any intervention to the female reproductive system—i.e. they require no medicines or devices.

| | |
|---|---|
| Coitus interruptus | Mucus approach |
| Fertility awareness | (changes in uterine secretion) |
| Calendar-based method | Changes in the cervix |
| Basal temperature measurement | Saliva crystallization detected |
| Determining levels of sexual | by microscope |
| hormones | LH, FSH, E as a contraceptive |
| | method |

Every method and from of these contraceptive methods can be used effectively to determine the ovulatory phase in particular. The initial phase and the terminal phase of this period are extremely unstable; therefore they cannot satisfy requirements of determining the whole term of the ovulatory period.

### Barrier Methods

The aim of these methods is to eliminate defects of natural methods by mechanical barriers. They are applied both in men and women.

| | |
|---|---|
| Condom | Cervical cap |
| Diaphragm/applied in women | Female condom - femidom |

### Chemical Contraception

This is used to destroy sperm during sexual intercourse.

| | |
|---|---|
| Spermicide | Contraceptive sponge |

Each of these methods is more or less reliable ranging from 72% for natural methods up to 88% for condom.

### Intrauterine Devices

The device acts as a foreign object in the uterus, creating a long-term or even chronic inflammatory process. This results in intensive defense activities—high leukocyte production that kills sperm.

### Hormonal Contraception

Its active ingredients present the highest threat for future generations of the mankind.

Function and importance of hormones—estrogens in a female organism is very extensive, as these hormones have effects in more than 300 tissues.

Mechanism of function and effects of biosynthesis of reproductive hormones in a female organism is based on the principle of negative feedback between the ovaries and hypothalamus and pituitary gland. Steroids (reproductive and adrenal hormones) neither are nor produced as a reserve.

The function and desired effects of synthetic hormonal contraceptives is based on this principle by delivering and maintaining enormous amounts of oestrogen and progesterone everyday in the female organism. This creates a high (higher by several thousand %) daily supply compared to their natural physiological need, presenting an extremely high risk of development of genetic disorders in children, in second and third generations in particular.

In this process, the amount of synthetic hormone supplies is 75-200 μg for progesterone and 15-35 μg for oestrogen irrespective of their form and application method.

A comparison of oestrogen production in a woman in 24 hours by body's own biosynthesis and an everyday supply of synthetic hormones to the body gives alarming or even catastrophic results after conversion: The female organism usually secrets 5.22 μg of oestrogen during the ovulatory period in 24 hours and 1.36 μg outside the ovulatory period.

An everyday application of synthetic contraceptives results on undesired, harmful and abnormal levels—13 200 000 up to 33 200 000 pg, exceeding the limit of physiological need of the daily supply. When these undesired and forbidding daily levels are expressed in %, a calculation or the ovulatory period exceeds the physiological need by 340 to 810%. For non-ovulatory period, this increase is alarmingly dangerous—it is higher by 1 122 to 2 573%.

Actually, women taking contraceptives do not ovulate because their reproductive organs are not functioning (as if they were in coma), so they affect the female organism continuously at the level of the non-ovulatory period.

The result is that any functional and effective activities of natural hormones of the reproductive system and adrenal glands have to be ensured by synthetic hormones.

This means that all the steroid hormones are made from synthetic hormones. As steroids are known to be incorporated by their effects directly to the cell nuclei—DNA then to RNA, and affect protein composition in cells therefore in organs, too, a detailed review of this fact and its possible consequences shows that steroids have effects at the genetic level and affect function of the body.

Considering this issue, it is necessary to become familiar with the reality that effects of contraceptives have some minor benefits, but bring many medical disorders and even more risks.

### Possible Medical Risks

### Adverse Effects:

| | |
|---|---|
| Blood clotting | Weight gain |
| Myocardial infraction (heart stroke) | Livers diseases |
| Metabolic changes - lipids, glucose | Breast and cervical tumours |
| Benign tumours | |

It is also known that sexual hormones have distinct psychological effects manifested for example during puberty, but especially during a long-term administration of synthetic oestrogen- and progesterone-based contraceptives.
This creates all the conditions needed for accelerated aging of the entire reproductive system (it is in coma)—menopause.

### Side Effects

| | |
|---|---|
| Depression | Photosensitivity - red marks |
| Change in sexual activity | from sun exposure |
| Nausea and vomiting | More body hair |
| Headache | Higher blood pressure |
| Vision disorders | Hair more oily |
| Premenstrual syndrome | Higher flatulency |
| Changes in vaginal secretion | Breasts appear bigger and |
| Chloasma - pigmented marks on | are more sensitive |
| the face | Milk discharged from breasts |
| | Convulsions in legs |

### Medical Benefits

| | |
|---|---|
| Pregnancy prevention | Probable decrease of uterine myoma |
| Protection from ovarian | Painful menstruation relieved |
| inflammation | Prevention for ovarian and uterine |
| Protection for ovarian cysts | cancer |

These organs actually cannot catch a disease as they are in coma and defunct, so no medical benefits can be there; the benefits are speculative only.

### BIBLIOGRAPHY

Radim UZEL: Jak neot\check{e}hotn\check{e}t, SCIENTIKA MEDIA, PRAGUE 1992
Ji\check{r}i PRESL et al.: Kontracepce, GALÉN, PRAGUE 1993
Anne SZAREVSKÁ, John GUILLEBAUD: Antikoncepce, VIKTORIA PUBLISHING, a.s. PRAGUE 1996
Elaborates promoting synthetic hormonal contraception at gynaecologists, 2004-2007
a Combined Aid to Determine the Optimum Time for Fertilization in Women from Urine

This aid belongs to natural contraceptive methods because ovulation is determined using a test tube with a sample of urine outside the female body. The determination by analysis is very simple and sufficiently fast—it takes only 15 minutes or 30 in maximum. This method is invention of first inventor, but after 20 years it is not covered by a patent protection (Patent SK No. 279 304).

With regard to this, it has to be said that the ovulatory period is a stable physiological process that creates identical biochemical processes and their respective metabolites in every healthy female organism.

Compositions of the solution (covered by the invention) are therefore given by these processes and create identical or similar manifestations just like ovulatory period indicators or ovulation indicators do.

Further development and improvement of this method as a contraceptive required development of a single-ingredient solution compared to a combined solution.

The carrier—casein in the combined solution was not able to bind with the anion (sulphate —SO₄) because a chemical reaction—precipitation occurred immediately upon contact. To produce a single-ingredient solution it was necessary to find an appropriate—optimum anion (with corresponding kations and acids) that would permanently sustain the mutual bond with the carrier.

### SUMMARY OF INVENTION

The aforementioned disadvantages of specific methods of contraception and the current state of technology eliminate the use of the solution covered by the invention to determine the entire ovulatory period in women from a test tube of urine (without any intervention to the female organism), the essence of which is that the solution contains from 0.5 to 9.0% by mass of hydrate salt of chloride anion Cl⁺ with a chemical element of bivalent kation of cobalt or copper, magnesium, manganese, zinc, calcium or a monovalent kation of sodium, potassium; 0.5 to 10.5% by volume of oxalic acid, formic acid, acetic acid, butyric acid, citric acid, lactic acid, hydrofluoric acid, boric acid, tartaric acid; 0.2 to 8.0% by mass of casein and 80.5 to 98.5% by volume of water. The overall pH of the solution is 0.8 to 3.5%.

The invention presents distinctive benefits compared to the current state of technology. The solution includes a single ingredient (more components). It can by used by any woman having a healthy sexual reproduction cycle; samples are easy to take for analysis and the analytical procedure is simple without any apparatus or equipment. The analysis can be done immediately after taking a sample without any treatments or requirements for special rooms and qualifications. A single piece of information by reading or word is sufficient. Results of the analysis are unbiased without any subjective doubts and they are known in 5 minutes, maximum in 30 minutes. The test is qualitative.

The effect of the solution is manifested by a direct bond of free sulphate SO₄²⁻ anions in a sample of urine to the Cl⁺ anion, cation, acid and the carrier, added by the solution covered by the invention, in an acidic environment. Effects of components of the solution will form a precipitate in this reaction.

Presence of free sulphate SO₄²⁻ anions in urine during the test can be seen in dispersion of the initial precipitate in the liquid along the whole test tube and this precipitate starts to float with increasing amounts of free sulphate anions in urine. If the sulphate SO₄²⁻ anions are bound with kations in the urine, the precipitate descends to the bottom of the test tube.

Besides formulation of the solution, the invention brings additional benefits.

The greatest benefit of the invention is identification of the optimum anion-chloride Cl⁺ that is combined with the carrier (casein) at the time of manufacture (reconstitution) of the solution, but also permanently with corresponding cations and acids. This ensures that all the ingredients of the solution are added to a single complete set—i.e. they create a single-ingredient solution used to determine ovulatory period in women from a test tube of urine. This increases its reliability and allows concurrent effects of every solution ingredient during analysis. This improves qualitative level of results of the analysis and avoids errors in the order of solutions during analysis.

In a healthy female organism, the bond between some kations and anions of sulphate SO₄²⁻ is broken at the time of ovulation.

This condition is supported by biochemical changes in the body and affects successful fertilization, and this where effects of free SO₄²⁻ anions and some kations on these processes and subsequent pregnancy can be seen especially in the sulphate-kation bond, thus in transfer of ions into cells by means of aldosterone, a hormone released in higher amounts during ovulation and during the entire period of pregnancy.

This specifies and extends another significant function of these bivalent kations in the field of reproduction. So far, they have been defined only in terms of health and they have been said to be important and needed for reproduction.

Investigation of the optimum combination—bonds of the solution covered by the invention resulted in development of a new method of contraception that has become permanently established for optimum determination of ovulatory period in women, as BIOCHEMICAL METHOD OF CONTRACEPTION.

This is possible due to its properties—high effectiveness and reliability and mainly due to the fact that this is the only method (at present) that satisfies all the requirements of optimum contraception (in global terms) for every group of women—in terms of health, social status, religion, age etc.

The method is optimal because it uses and determines biochemical indicators (metabolites) resulting from physiological changes in the female body at the ovulatory period, i.e. the metabolites become the basic active substance—material for the significant working method.

Biochemical metabolites of the ovulatory load in the female body are the most optimum products for determining the ovulatory period. Compared to any other existing method for determining ovulation (at present), they are the most reliable and allow to determine the entire ovulatory period by the most objective and most distinct manifestation of assessment test, i.e. objective manifestation of phases: initial—main ovulatory—final.

The biochemical method of contraception is—a unique, outstanding, pioneering, brand new contraceptive method developed from knowledge from research findings. In global terms, this method (method of biochemical metabolites) has not been used yet at all.

The present invention, being a new, comprehensive complete contraceptive method, has characteristics of a discovery for women as the only optimum method of contraception for women in global terms.

### BEST MODE OF CARRYING OUT THE INVENTION

The invention will be designed specifically as a solution, where −1000 ml of the solution contains:
20 g (2.0% by mass) of hydrate salt of cobalt (II) chloride
80 ml (8.0% by volume) of oxalic acid
20 g (2.0% by mass) of casein
880 ml (88.0% by volume) of water
pH of the solution is 0.8
35 g (3.5% by mass) of hydrate salt of calcium chloride
24 ml (2.4% by volume) of acetic acid
80 g (8.0% by mass) of casein
861 ml (86.1% by volume) of water
pH of the solution is 2.4
90 g (9.0% by mass) of hydrate salt of magnesium chloride
31 ml (3.1% by volume) of butyric acid
72 g (7.2% by mass) of casein
807 ml (80.7% by volume) of water
pH of the solution is 3.5
5 g (0.5% by mass) of hydrate salt of manganese chloride
18 ml (1.8% by volume) of formic acid
63 g (6.3% by mass) of casein
914 ml (91.4% by volume) of water
pH of the solution is 1.9
72 g (7.2% by mass) of hydrate salt of sodium chloride
44 ml (4.4% by volume) of lactic acid
14 g (1.4% by mass) of casein
870 ml (87.0% by volume) of water
pH of the solution is 2.7
25 g (2.5% by mass) of hydrate salt of zinc chloride
92 ml (9.2% by volume) of tartaric acid
51 g (5.1% by mass) of casein
832 ml (83.2% by volume) of water
pH of the solution is 3.0
18 g (1.8% by mass) of hydrate salt of calcium chloride
72 ml (7.2% by volume) of citric acid
46 g (4.6% by mass) of casein
864 ml (86.4% by volume) of water
pH of the solution is 2.6
84 g (8.4% by mass) of hydrate salt of copper chloride
67 ml (6.7% by volume) of boric acid
32 g (3.2% by mass) of casein
817 ml (81.7% by volume) of water
pH of the solution is 3.1
50 g (5.0% by mass) of hydrate salt of sodium chloride
105 ml (10.5% by volume) of hydrofluoric acid
2.0 g (0.2% by mass) of casein
843 ml (84.3% by volume) of water
pH of the solution is 2.8
46 g (4.6% by mass) of hydrate salt of potassium chloride
57 ml (5.7% by volume) of formic acid
27 g (2.7% by mass) of casein
870 ml (87.0% by volume) of water
pH of the solution is 2.4
62 g (6.2% by mass) of hydrate salt of magnesium chloride
5.0 ml (0.5% by volume) of lactic acid
58 g (5.8% by mass) of casein
875 ml (87.5% by volume) of water
pH of the solution is 2.9

The examples above are just one of the numerous possible combinations of the total number of possible combinations of hydrate salt of chloride Cl+ anion with the mentioned chemical components in bivalent or monovalent kations and specific acids in terms of their percentage range.

Feasibility of Test with the Solution Covered by the Invention

Put 2.0 ml of urine into a test tube, add 1.0 ml of the solution covered by the invention and mix 2-3 times by reverting the test tube by 180°. Put the test tube to the vertical position to avoid it from turning and to allow dispersion of the precipitate to a relevant position for 5 minutes or 30 minutes in maximum to determine the result of the test.

The result is evaluated according to the position of the precipitate formed in the test tube:
The precipitate descends to the bottom of the test tube—no fertilisation;
The precipitation does not descend and does not float in the liquid—it is dispersed along the liquid—appropriate time for fertilisation begins (a clear liquid is usually seen at the bottom of the solution), it lasts 1 day or ends (a clear liquid is usually seen at the top of the solution)—lasts 1 day, too. However, fertilisation might happen in both cases—due to egg and sperm lifecycles;
The precipitate floats at the top of the liquid in the test tube—time appropriate for fertilisation—lasts 2 days.

During analysis, the solution works on the qualitative level, so accuracy of analysis is simpler (minor deviations in dosing have no impact on results) than in quantitative analyses.

Special monitoring of woman's own ovulatory cycles (not menstrual cycles) in individual months makes it possible for every woman to prevent undesired pregnancy. The product allows every woman to become familiar and monitor her own physiological process of ovulatory period and its validity as needed.

These facts create maximum possibilities to reduce numbers of forced induced abortions.

### INDUSTRIAL APPLICABILITY

Based on the invention, the solution should be used primarily to determine course of ovulatory cycles in individual months in women, with major focus on determining their ovulatory period as an appropriate time for fertilisation, but mostly as a protection against conception. This allows a personal and very simple monitoring of qualitative physiological course of the ovulatory period according to personal needs and to choose necessary treatment in case of any deviations.

It can be used also for detection of early pregnancy for information purposes by checking existence of the following ovulatory period.

The possibility of determining a valid ovulatory period should be verified carefully especially in adolescent girls as a prevention, where possible and early treatment can help prevent serious disorders that are difficult to treat in adult age, or that can be treated only partially.

The solution covered by the invention allows a successful monitoring of onset and course of menopause, where only the first stage of the ovulatory period is manifested—the analysed precipitate is dispersed only, but it does not float, i.e. there is no ovulation—no egg is produced.

To summarise, it can be said that the invention contributes to preservation of health in the mankind (against contraceptives).

1. (canceled)

2. A solution for determining the ovulatory period in a sample of urine, wherein 1000 ml of the solution contains from 5 to 90 g of hydrate salt of chloride anion Cl with a chemical element of bivalent cation of cobalt, copper, magnesium, manganese, zinc, calcium or a monovalent cation of sodium or potassium; from 5 to 105 ml of oxalic acid, formic acid, acetic acid, butyric acid, citric acid, lactic acid, hydrofluoric acid, boric acid or tartaric acid; from 2 to 80 g of casein and up to 1000 ml water, and wherein the pH of the solution is 0.8 to 3.5.

3. A method for determining the ovulatory period in a sample of urine comprising the following steps:
adding the sample of urine to a test tube;
adding a solution wherein each 1000 ml of the solution contains from 5 to 90 g of hydrate salt of chloride anion Cl with a chemical element of bivalent cation of cobalt, copper, magnesium, manganese, zinc, calcium or a monovalent cation of sodium or potassium; from 5 to 105 ml of oxalic acid, formic acid, acetic acid, butyric acid, citric acid, lactic acid, hydrofluoric acid, boric acid or tartaric acid; from 2 to 80 g of casein and up to 1000 ml water, and wherein the pH of the solution is 0.8 to 3.5; and
assessing the precipitate after 5-30 minutes;
wherein, if
a. the precipitate descends to the bottom of the test tube then no fertilisation can occur;
b. the precipitation does not descend and does not float in the liquid then fertilisation begins; and if
c. the precipitate floats at the top of the liquid in the test tube that the time is appropriate for fertilisation.

4. The method of claim 3, wherein if the precipitation does not descend and does not float in the liquid and a clear liquid is seen at the bottom of the solution, the time for fertilization lasts one day or ends and if a clear liquid is seen at the top of the solution the time for fertilization also lasts one day.

5. The method of claim 3, wherein if the precipitate floats at the top of the liquid the time for fertilization lasts 2 days.
